(19) United States
(12) Patent Application Publication
(10) Pub. No.: US 2023/0233449 A1
(43) Pub. Date: Jul. 27, 2023

(54) A CREAM COMPOSITION WITH HERBAL EXTRACT AND PRODUCTION METHOD THEREOF

(71) Applicant: SBS BILIMSEL BIOÇÖZÜMLER SANAYI VE TICARET ANONIM SIRKETI, Istanbul (TR)

(72) Inventor: Asli Elif TANUGUR SAMANCI, Instanbul (TR)

(21) Appl. No.: 18/003,540

(22) PCT Filed: Nov. 26, 2021
(86) PCT No.: PCT/TR2021/051304

§ 371 Date: Dec. 28, 2022

(30) Foreign Application Priority Data
Dec. 18, 2020 (TR) 2020/20931

(51) Int. Cl. A61K 8/98; A61Q 19/00

(52) U.S. Cl. CPC A61K 8/988; A61Q 19/00; A61K 2800/805

(57) ABSTRACT

Disclosed is a cream composition with herbal extract for the face, body, hand, foot and joint developed to be used in the cosmetic industry for the treatment of skin diseases, aging, joint pain, wrinkles and various wounds, and to the production method thereof. The cream composition includes at least one of bee venom, propolis, royal jelly and honey as active ingredients, water, stearyl alcohol, acholine GC, lipex shealight, jojoba oil, bentonite, shea butter, beeswax, hydrogenated lecithin or olive oil as other ingredients. A disclosed production method includes the steps of heating the oil phase, cooling the oil phase, adding the bee venom phase, homogenization and adding propolis.

### FIELD OF THE INVENTION

The present invention relates to a cream composition to be used as a face, body, hand, foot or joint cream and its production method containing completely natural components in addition to bee venom, propolis, royal jelly and honey.

In particular, the present invention relates to a cream composition with herbal extract for the face, body, hand, foot and joint developed to be used in the cosmetic industry for the treatment of skin diseases, aging, joint pain, wrinkles and various wounds, and to the production method thereof.

### STATE OF THE ART

Today, there are creams containing bee venom on the market. When their content is examined, it has been observed that only bee venom is used as the active ingredient or that there are various chemicals in their contents.

When the creams containing bee venom in the state of the art are examined, it has been observed that only bee venom is used as the active ingredient or that there are various chemicals in their contents. In addition, there can be drugs in cell regenerating and pain-relieving creams in the products of the state of the art. However, it is possible to eliminate harmful effects on health by using herbal and natural products instead of these. For this reason, it is intended in the subject of the invention to increase the effectiveness of the cream by adding propolis, royal jelly, honey and beeswax from other bee products to the formulation in addition to bee venom.

The application numbered RU2163118C1 has been encountered in literature. Here, a massage cream containing Bee-glue wax, dimexide, horse radish infusion, thick bee glue extract, bee infusion or bee venom vegetable oils and camphor oil is disclosed. Dimethyl sulfoxide (dimexide) contained in the patent is a drug with analgesic and antiseptic effects. Thus, it was used for massage in case of rheumatoid arthritis and osteoarthritis together with bee venom. On the contrary, the inventive cream does not contain any drugs or chemicals in its composition and it is prepared with completely natural ingredients. While the anti-inflammatory effect is increased with royal jelly and honey, which are not included in the patent content but used within the scope of the present invention, it is also effective for various skin problems such as wounds and eczema. In addition, various oils, glycerin and vitamin E within the scope of the present invention provide natural moisturizing and nourishment to the skin.

The application numbered US20120128784A1 has been encountered in literature. Here, a composition that shows antibacterial activity against acne-causing bacteria and which has been developed to prevent and treat acne formation is disclosed. The formulation of nourishing cream containing bee venom within the scope of the application comprises bee venom, sitosterol, polyglycerin 2-oleate, ceramide, stearate, cholesterol, dysethyl phosphate, glycerin, sunflower oil, carboxyvinyl polymer, xanthan gum, Triethanolamine, perfume and water. Triethanolamine is used as a moisturizes in the content here. This component, which is used in creams for its moisturizing properties, has carcinogenic effects. On the other hand, such components are not included in our present invention. The moisturizing effect in our present invention is provided with glycerin and natural oils. In addition, various skin problems such as wounds, inflammation and eczema have been solved with propolis, royal jelly and honey, which are not included in the patent, but are found in our present invention. In addition to the effects of bee venom, the components required by the skin are provided by using various oils, vitamin E and beeswax in scope of present invention.

As a result, due to the abovementioned disadvantages and the insufficiency of the current solutions regarding the subject matter, a development is required to be made in the relevant technical field.

### BRIEF DESCRIPTION OF THE INVENTION

The present invention relates to a cream composition and production method thereof which fulfills the abovementioned requirements, eliminates all disadvantages and brings some additional advantages.

The main aim of the invention is to develop a cream composition whose effectiveness has been increased with bee products such as propolis, royal jelly, honey and beeswax in addition to bee venom and which has natural ingredients with no harmful effects on health.

A similar aim of the invention is to develop a cream composition which is effective and beneficial on the skin by means of the bee products and provides solutions to various skin problems such as wounds, inflammation and eczema with the anti-inflammatory effect increased by propolis, royal jelly and honey.

Another aim of the invention is to develop a cream composition which provides protection against various microbiological deteriorations with natural origin content by the antifungal, antimicrobial and antiviral properties of propolis and bee venom contained therein.

Another aim of the invention is to develop a cream composition which moisturizes the skin and nourishes it with the components it needs with natural content by means of the various oils it contains, glycerin and vitamin E.

In order to fulfill the above-mentioned aims, the inventive cream composition of the invention comprises at the one of the components; bee venom, propolis, royal jelly and honey as active ingredients, water, stearyl alcohol, acholine GC, lipex shealight, jojoba oil, bentonite, shea butter, beeswax, hydrogenated lecithin or olive oil as other ingredients the inventive production method comprises the steps of heating the oil phase, cooling the oil phase, adding the bee venom phase, homogenization and adding propolis.

The structural and characteristic features of the present invention will be understood clearly by the following detailed description and therefore the evaluation shall be made by taking the detailed description into consideration.

### DETAILED DESCRIPTION OF THE INVENTION

In this detailed description, the inventive cream composition with herbal extract and the production method thereof is described only for clarifying the subject matter in a manner such that no limiting effect is created.

The inventive cream composition contains bee venom, propolis, royal jelly and honey as an active ingredient. This cream composition contains at least one of olive oil, jojoba oil, shea butter, bentonite, stearyl alcohol or acholine GC, carob bean gum, NaCl, beeswax, glycerin or vitamin E as other ingredients.

In the inventive cream composition bee venom is used as a natural product with antibacterial, antifungal, anti-inflammatory, nervous system regulating, painkiller and anti-aging effects so as to solve skin diseases such as wrinkles, joint pains, spot and acne. On one hand, propolis is used so as to heal wounds on the skin and to provide natural protection to the skin with its chemical content. Royal jelly is used to provide protection from UV rays in addition to reducing the signs of aging by supporting collagen production with its antibacterial activity, anti-inflammatory activity, vasodilative and hypotensive activities, disinfectant action and antioxidant activity. On the other hand, honey has a healing effect on skin diseases and wounds such as wounds, eczema and inflammation on the skin. Besides it is used in the inventive cream composition in order to strengthen the skin immune system by inhibiting the growth of dermatologically important microorganisms.

Carob bean gum which is used in the preferred embodiments of the inventive cream composition ensures that viscosities of cream formulations are brought to the desired consistency. Lipex shealight provides moisturizing, softening effect and care by nourishing the skin in the invention. Jojoba oil is used to have a softening effect on the skin in addition to its benefit in curing skin diseases such as eczema. Vitamin E is used in the inventive cream composition so as to present a protective and regenerative feature on the skin with its high antioxidant effect. Jasmine oil, which is an essential oil, is used instead of fragrant chemicals so as to provide a pleasant and light fragrance in the cream composition. In order to increase the viscosity naturally, the texture of the creams is thickened by using NaCl, which has volume-enhancing and masking properties. Beeswax, which is also a natural bee product, is used to increase the viscosity of the cream composition. Olive oil is used as an emollient in cream composition, with its ability to be easily absorbed by the skin and nourishing. Akoline GC derived from vegetable oils is a natural emulsifier that provides tightness and smoothness to the skin. For these reasons, it is used in the inventive cream composition so as to exhibit softening, nourishing and balancing properties. Stearyl alcohol is also used as an emulsifier in cream composition. Bentonite is used both as a natural preservative and as an emulsion in the inventive cream composition.

The inventive cream composition can be a face, body, hand, foot and joint cream. The composition of the cream intended to be used for the face mainly contains deionized water, royal jelly, honey, bee venom, stearyl alcohol, akoline GC, lipex shealight, jojoba oil and propolis extract. A preferred embodiment of said face cream composition additionally contains carob bean gum, glycerin, vitamin E and jasmine oil. The weight amounts of the components of this preferred embodiment developed to be used in percent are presented in Table 1.

TABLE 1
The preferred formulation of the cream
composition developed for face

| | Amount preferred | Usable amount |
|---|---|---|
| | by weight (%) | by weight (%) |
| Deionized Water | 79.40 | 75-80 |
| Carob Bean Gum | 0.25 | 0.1-0.3 |
| Glycerine | 2.00 | 1.5-2.5 |
| Royal Jelly | 1.00 | 0.8-1.5 |
| Honey | 0.45 | 0.25-1.50 |
| Bee Venom | 0.10 | 0.05-0.15 |
| Stearyl Alcohol | 3.00 | 1-5 |
| Acholine GC | 3.00 | 1-5 |
| Lipex Shealight | 7.00 | 5-8 |
| Jojoba Oil | 2.00 | 1-5 |
| Vitamin E | 0.50 | 0.1-0.8 |
| Propolis Extract | 0.30 | 0.1-0.5 |
| Jasmine Oil | 1.00 | 0.5-1.5 |

The cream composition intended to be used for the body mainly comprises deionized water, royal jelly, honey, bee venom, stearyl alcohol, acholine GC, lipex shealight, jojoba oil and propolis extract. A preferred embodiment of said body cream composition additionally contains carob bean gum, glycerin, vitamin E and jasmine oil. The weight amounts of the components of this preferred embodiment developed to be used in body are presented in Table 2 in percentage.

TABLE 2
The preferred formulation of the cream
composition developed for body

| | Amount preferred | Usable amount |
|---|---|---|
| | by weight (%) | by weight (%) |
| Deionized Water | 77.40 | 75-80 |
| Carob Bean Gum | 0.25 | 0.1-0.3 |
| Glycerine | 2.00 | 1.5-2.5 |
| Royal Jelly | 1.00 | 0.8-1.5 |
| Honey | 0.45 | 0.25-1.50 |
| Bee Venom | 0.10 | 0.05-0.15 |
| Stearyl Alcohol | 3.00 | 1-5 |
| Acholine GC | 5.00 | 1-5 |
| Lipex Shealight | 5.00 | 5-8 |
| Jojoba Oil | 4.00 | 1-5 |
| Vitamin E | 0.50 | 0.1-0.8 |
| Propolis Extract | 0.30 | 0.1-0.5 |
| Jasmine Oil | 1.00 | 0.5-1.5 |

The cream composition intended to be used for foot mainly comprises deionized water, royal jelly, honey, bee venom, bentonite, jojoba oil, shea butter and propolis extract. A preferred embodiment of said foot cream composition additionally contains glycerine, NaCl and jasmine oil. The weight amounts of the components of this preferred embodiment developed to be used in foot are presented in Table 3 in percentage.

TABLE 3
The preferred formulation of the cream
composition developed for foot

| | Amount preferred | Usable amount |
|---|---|---|
| | by weight (%) | by weight (%) |
| Deionized Water | 69.60 | 65-80 |
| Glycerine | 3.00 | 1-5 |
| NaCl | 0.50 | 0.1-0.8 |
| Royal Jelly | 1.00 | 0.8-1.5 |
| Honey | 1.00 | 0.25-1.50 |
| Bee Venom | 0.10 | 0.05-0.15 |
| Bentonite | 8.00 | 5-10 |
| Jojoba Oil | 4.00 | 1-5 |
| Shea Butter | 12.00 | 5-15 |
| Propolis Extract | 0.30 | 0.1-0.5 |
| Jasmine Oil | 0.50 | 0.5-1.5 |

The cream composition intended to be used for hand mainly comprises deionized water, royal jelly, honey, bee venom, bentonite, jojoba oil, shea butter and propolis extract. A preferred embodiment of said hand cream composition additionally contains glycerine, NaCl and jasmine oil. The weight amounts of the components of this preferred embodiment developed to be used in hand are presented in Table 4 in percentage.

TABLE 4
The preferred formulation of the cream
composition developed for hand.

| | Amount preferred | Usable amount |
|---|---|---|
| | by weight (%) | by weight (%) |
| Deionized Water | 69.60 | 65-80 |
| Glycerine | 3.00 | 1-5 |
| NaCl | 0.50 | 0.1-0.8 |
| Royal Jelly | 1.00 | 0.8-1.5 |
| Honey | 1.00 | 0.25-1.50 |
| Bee Venom | 0.10 | 0.05-0.15 |
| Bentonite | 7.00 | 5-10 |
| Jojoba Oil | 5.00 | 1-5 |
| Shea Butter | 12.00 | 5-15 |
| Propolis Extract | 0.30 | 0.1-0.5 |
| Jasmine Oil | 0.50 | 0.5-1.5 |

Instead of jasmine oil, natural lavender essential oil, natural rose oil or lemon balm oil are used in other preferred embodiments of the inventive cream composition intended for use on the face, body, feet or hands. Likewise, solid glycerine is used instead of liquid glycerine in different preferred embodiments. Also, xanthan gum is used instead of carob bean gum in preferred embodiments.

The cream composition intended to be used for joint mainly comprises beeswax, olive oil, royal jelly, honey, bee venom, shea butter and propolis extract. A preferred embodiment of said joint cream composition additionally contains jasmine oil. The weight amounts of the components of this preferred embodiment developed to be used in joints are presented in Table 5 in percentage.

TABLE 5
The preferred formulation of the cream
composition developed for joint

| | Amount preferred | Usable amount |
|---|---|---|
| | by weight (%) | by weight (%) |
| Beeswax | 14.10 | 10-15 |
| Shea Butter | 20.00 | 10-25 |
| Royal Jelly | 1.00 | 0.8-1.5 |
| Olive Oil | 58.50 | 50-70 |
| Honey | 5.00 | 3-10 |
| Bee Venom | 0.10 | 0.05-0.15 |
| Propolis Extract | 0.30 | 0.1-0.5 |
| Jasmine Oil | 1.00 | 0.5-1.5 |

Instead of jasmine oil, natural lavender essential oil, natural rose oil or lemon balm oil are used in other preferred embodiments of the inventive cream composition intended for use on joints. Likewise, hydrogenated lecithin is used instead of beeswax in different preferred embodiments.

In order to obtain the inventive cream composition, it mainly comprises the following method steps:
a. Heating of the oil phase,
b. Cooling of the oil phase,
c. Adding the bee venom phase,
d. Homogenizing,
e. Adding propolis,
f. Placing the final product in containers

The final product is in three types: soft for the face and body cream composition, medium for the hand and foot cream composition, and hard form for the joint cream composition. Preferred method steps for these different forms are described in detail below.

In the preferred production method to obtain the soft form of face and body cream composition, firstly, carob bean gum and water are mixed so as to obtain bee venom phase. The mixture is then heated in a heater in the range of 85-95° C. until the carob bean gum melts. The preferred temperature in this step is 90° C. After melting, the mixture is cooled to a temperature range of 45-50° C. in a cold water bath. Then, glycerin, honey, royal jelly and bee venom are added to the cooled mixture and mixed. Thus, the bee venom phase is obtained. Stearyl alcohol, alcoholine GC, lipex shealight and jojoba oil are heated with continuous stirring in a controlled manner until it reaches 50° C. in the heater in a separate container for the oil phase. When this oil phase is homogenized, it is taken from the heater and vitamin E and bee venom are added into the mixture. This mixture is then homogenized in a water bath at a speed of 1500-2000 rpm. In this step, the mixing speed is preferably 2000 rpm. Propolis and jasmine oil are finally added into this mixture and mixed, and the final product in soft form is obtained. This final product obtained is stored in containers.

In the preferred production method to obtain the hand and foot cream composition in medium form, bentonite, jojoba oil and shea butter are mixed and heated to a temperature range of 35-45° C. for oil phase. The preferred temperature in this step is 40° C. At the same time, deionized water, glycerin, NaCl, royal jelly, honey and bee venom are mixed in a separate container until they dissolve. When the oil phase melts, it is taken from the heater and cooled in a water bath. Afterwards, the bee venom phase is added into the oil phase when cooling takes place. Then, the mixture is then homogenized in a water bath at a speed of 1500-2000 rpm. In this step, the mixing speed is preferably 2000 rpm. When homogenization is performed, propolis and jasmine oil are added into the mixture and mixed. Thus, the final product in medium form is obtained. This final product obtained is stored in containers.

In the preferred production method to obtain the joint cream composition in hard form, beeswax, shea butter and olive oil are heated to a temperature range of 60-80° C. for the oil phase. The preferred temperature in this step is 70° C. Then, after the oil phase melts, it is taken from the heater and cooled in a water bath. Then, royal jelly, honey and bee venom are added into the oil phase. This mixture is then homogenized in a water bath at a speed of 1500-2000 rpm. In this step, the mixing speed is preferably 2000 rpm. When homogenization is performed, propolis and jasmine oil are added into the mixture and mixed. Thus, the final product in hard form is obtained. This final product obtained is stored in containers.

Bee venom, which is contained as an active ingredient in the inventive cream composition, is a natural bee product that can be used in cosmetics with its skin tightening and skin protective effects. In general, bee venom has antibacterial, anti-inflammatory, anti-aging, cell regenerative, nervous system regulatory and pain relief effects. In addition to its effects on the nervous system, it also has positive effects on diabetic and various skin diseases, it is also considered as an important substance for cancer treatment. It is used in skin diseases, as it is good for wounds and inflammations, and in the treatment of rheumatism and migraine with its pain relief feature. In addition to these, it is effective on epilepsy, chronic fatigue, vascular occlusion, asthma and regulation of cholesterol.

Propolis, which is contained as an active ingredient in the inventive cream composition, is a bee product with the feature of being a natural antibiotic. It provides many beneficial biological activities besides antioxidant, antimicrobial, antifungal, antivirus, anti-inflammatory, anticarcinogenic and anesthetic effects. It strengthens the immune system, is effective against cancer, inflammatory diseases, viral and bacterial diseases.

Royal jelly, which is included as an active ingredient in the inventive cream composition is known to have numerous functional properties such as antibacterial, anti-inflammatory activity, disinfectant action, antioxidant and antitumor activity, as well as being the food of bees. It is also known to protect the skin from UV rays, prevent wrinkles and prevent signs of aging on the skin by producing collagen.

Honey, which is contained as an active ingredient in the inventive cream composition is a bee product used to heal wounds on the skin. It is known that honey is used for the treatment of wounds, eczema and inflammations, as well as preventing the growth of microorganisms and affecting the immune system of the skin.

The diseased area should be thoroughly cleaned with a natural soap before applying the inventive cream composition for the treatment of skin diseases. Then, the preferred cream composition is applied by massaging to the face, body, hand, foot or joint areas according to the area of effect. The inventive preferred cream composition is applicable twice a day, in the morning and evening, until the diseased area is healed.

Since the invention contains bee venom, propolis, royal jelly and honey, it can cause allergic reactions in some people. For this reason, it is recommended to apply the test to a small area in the wrist and wait for 15 minutes before applying it to the diseased area.

### REFERENCES

1—S. Bogdanov, “Functional and biological properties of the bee products: a review,” Bee Product Science, pp. 1-12, 2011.
2—M. C. Kiernan, S. Vucic, B. C. Cheah, M. R. Turner, A. Einsen, O. Hardiman, J. M. Burrell, and M. C. Zoing, “Amyotrophic lateral sclerosis,” Lancet, vol. 377, no. 9769, pp. 942-955, 2011, doi: 10.1016/S0140-6736(10)61156-7.
3—E. J. Yang, J. H. Jiang, S. M. Lee, S. C. Yang, H. S. Hwang, M. S. Lee, and S. M. Choi, “Bee venom attenuates neuroinflammatory events and extends survival in amyotrophic lateral sclerosis models,” Journal of Neuroinflammation, vol. 7, no. 69, pp. 1-12, 2010.
4—H. Nazik, M. Turhan, T. T. Koca, M. K. Müllayim, P. Öztürk ve S. Nazik, “Aricilarda ari ürünlerine maruziyet ile allerji, inflamasyon ve enfeksiyon ilişkili durumlarin araştirilmasi,” KSÜ Tip Fakültesi Dergisi, c. 13, s. 2, ss. 34-37, 2018.
5—S. M. Han, K. G. Lee, J. H. Yeo, and S. C. Pak, “Dermal and ocular irritation studies of honeybee (Apis mellifera I.) venom,” The American Journal of Chinese Medicine, vol. 40, no. 4, pp. 795-800, 2012, doi: 10.1142/S0192415X12500590.
6—E. Birben ve C. Saçkesen, “Allerjik enflamasyon,” Çocuk Sağliği ve Hastaliklari Dergisi, c. 57, s. 1, ss. 35-49, 2014.
7—A. G. Hegazi, F. A. A. Raboh, N. E. Ramzy, D. M. Shaaban, and D. Y. Khader, “Bee venom and propolis as new treatment modality in patients with localized plaque psoriases,” International Research Journal of Medicine and Medical Science, vol. 1, no. 1, pp. 27-33, 2013.
8—S. Han, K. Lee, J. Yeo, W. Kim, and K. Park, “Biological effects of treatment of an animal skin wound with honeybee (Apis melifera. L) venom,” Journal of Plastic, Reconstructive & Aesthetic Surgery, vol. 64, no. 3, pp. e67-e72, 2011, doi: 10.1016/j.bjps.2010.08.022
9—K. Kim, W. Lee, H. An, J. Kim, H. Chung, S. Han, M. Lee, K. Lee, S. C. Pak, and K. Pak, “Bee venom ameliorates compound 48/80-induced atopic dermatitis-related symptoms,” International Journal of Clinical and Experimental Pathology, vol. 6, no. 12, pp. 2896-2903, 2013.
10—H. An, W. Lee, K. Kim, J. Kim, S. Lee, S. Han, K. Lee, C. Lee, and K. Park, “Inhibitory effects of bee venom on Propionibacterium acnes—induced inflammatory skin disease in an animal model,” International Journal of Molecular Medicine, vol. 34, no. 5, pp. 1341-1348, 2014, doi: 10.3892/ijmm.2014.1933.
11—S. Jeon, N. H. Kim, B. S. Koo, H. J. Lee, and A. Y. Lee, “Bee venom stimulates human melanocyte proliferation, melanogenesis, dendricity and migration,” Experimental and Molecular Medicine, vol. 39, no. 5, pp. 603-613, 2007, doi: 10.1038/emm.2007.66.
12—Y. G. Kim, M. Sumiyoshi, M. Sakanaka, and Y. Kimura, “Effects of ginseng saponins isolated from red ginseng on ultraviolet B-induced skin aging in hairless mice,” European Journal of Pharmacology, vol. 602, no. 1, pp. 148-156, 2009, doi: 10.1016/j.ejphar.2008.11.021.
13—C. S. Choi, J. K. Park, C. G. An, E. H. Lee, and K. J. Ahn, “Multiple benefits of bee venom in aged skin manifestations,” Kor J Aesthet Cosmetol, vol. 13, no. 5, pp. 679-688, 2015.
14—S. M. Han, I. P. Hong, S. O. Woo, S. N. Chan, K. K. Park, Y. M. Nicholls, and S. C. Pak, “The beneficial effects of honeybee-venom serum on facial wrinkles in humans,” Clinical Interventions in Aging, vol. 10, pp. 1587-1592, 2015, doi: 10.2147/CIA.S84940.
15—H. Hu, D. Chen, Y. Li, and X. Zhang, “Effect of polypeptides in bee venom on growth inhibition and apoptosis induction of the human hepatoma cell line SMMC-7721 in-vitro and Balb/c nude mice in-vivo,” Journal of Pharmacy and Pharmacology, vol. 58, no. 1, pp. 83-89, 2006, doi: 10.1211/jpp.58.1.0010.
16—M. Abdel-Rahman, A. S. Elebiary, S. S. Hafez, H. E. Mohammed, and A. E. Abdel Moneim, “Therapeutic activity of BEE-stings therapy in rheumatoid arthritis causes inflammation and oxidative stress in female patients,” International Journal of Research In Ayurveda and Pharmacy, vol. 4, no. 3, 2013, doi: 10.7897/2277-4343.04303.
17—N. H. Özdağ, S. Mollahaliloğlu, D. Öztş, G. B. Güzeldemirci, “Ağri tedavisinde akupunkturun yeri,” Ankara Medical Journal, vol. 15, no. 4, pp. 249-253, 2015, doi: 10.17098/amj.43740.
18—M. S. Lee, M. H. Pittler, B. Shin, J. C. Kong, and E. Ernst, “Bee venom acupuncture for musculoskeletal pain: a rewiew,” The Journal of Pain, vol. 9, no. 4, pp. 289-297, 2008, doi: 10.1016/j.jpain.2007.11.012.
19—A. Nitecka-Buchta, P. Buchta, E. Tabenska-Bosakowska, K. Walczynska, and S. Baron, “Myorelaxant effect of bee venom topical skin application in patients with RDC/TMD la and RDC/TMD lb: a randomized, double blinded study,” Biomed Research International, vol. 2014, pp. 9-12, 2014.
20—E. Zengin ve I. Karaca, “Böceklerin ilaçolarak kullanilmasi,” ADÜ Ziraat Dergisi, c. 14, s. 1, ss. 71-78, 2017, doi: 10.25308/aduziraat.294924
21—Ö. Çölkesen, “Apiterapi nedir,” Dozaj, c. 14, s. 4, ss. 7-11.
22—S. Bogdanov, “Bee venom: composition, health, medicine: a review,” Bee Product Science, pp. 1-24, 2017.

1. A cream composition with herbal extract, comprising, at least one of the following components: bee venom, propolis, royal jelly and honey as active ingredients, water, stearyl alcohol, acholine GC, lipex shealight, jojoba oil, bentonite, shea butter, beeswax, hydrogenated lecithin or olive oil as other ingredients.

2. A cream composition according to claim 1, wherein the cream composition is soft for use on the face or body, medium for hand or foot use, or hard for joint use.

3. A cream composition according to claim 2, comprising, in soft and medium form, solid or liquid glycerin in addition to honey, royal jelly, bee venom and water for the bee venom phase.

4. A cream composition according to claim 2, comprising, in soft form, carob or xanthan gum in addition to honey, royal jelly, bee venom and water for the bee venom phase.

5. A cream composition according to claim 2, comprising, in medium form, NaCl in addition to honey, royal jelly, bee venom and water for the bee venom phase.

6. A cream composition according to claim 1, comprising vitamin E.

7. A cream composition according to claim 1, comprising, the following by weight: deionized water in the range of 75-80%, carob bean gum in the range of 0.1-0.3%, glycerine in the range of 1.5-2.5%, royal jelly in the range of 0.8-1.5%, honey in the range of 0.25-1.5%, bee venom in the range of 0.05-0.15%, stearyl alcohol in the range of 1-5%, acholine gc in the range of 1-5%, lipex shealight in the range of 5-8%, jojoba oil in the range of 1-5%, Vitamin E in the range of 0.1-0.8%, propolis extract in the range of 0.1-0.5% and jasmine oil in the range of 0.5-1.5%.

8. A cream composition according to claim 7, comprising, the following by weight: deionized water in the ratio of 79.4%, carob bean gum in the ratio of 0.25%, glycerine in the ratio of 2%, royal jelly in the ratio of 1%, honey in the ratio of 0.45%, bee venom in the ratio of 0.1%, stearyl alcohol in the ratio of 3%, acholine gc in the ratio of 3%, lipex shealight in the ratio of 7%, jojoba oil in the ratio of 2%, vitamin E in the ratio of 0.5%, propolis extract in the ratio of 0.3% and jasmine oil in the ratio of 1%.

9. A cream composition according to claim 7, comprising, the following by weight: deionized water in the ratio of 77.4%, carob bean gum in the ratio of 0.25%, glycerine in the ratio of 2%, royal jelly in the range of 1%, honey in the ratio of 0.45%, bee venom in the ratio of 0.1%, stearyl alcohol in the range of 3%, acholine gc in the ratio of 5%, lipex shealight in the ratio of 5%, jojoba oil in the ratio of 4%, vitamin E in the ratio of 0.5%, propolis extract in the ratio of 0.3% and jasmine oil in the ratio of 1%.

10. A cream composition according to claim 1, comprising, the following by weight: deionized water in the range of 65-80%, glycerine in the range of 1-5%, NaCI in the range of 0.1-0.8%, royal jelly in the range of 0.8-1.5%, honey in the range of 0.25-1.5%, bee venom in the range of 0.05-0.15%, bentonite in the range of 5-10%, jojoba oil in the range of 1-5%, shea butter in the range of 5-15%, propolis extract in the range of 0.1-0.5% and jasmine oil in the range of 0.5-1.5%.

11. A cream composition according to claim 10, comprising, the following by weight: deionized water in the ratio of 69.6%, glycerine in the ratio of 3%, NaCI in the ratio of 0.5%, royal jelly in the ratio of 1%, honey in the ratio of 1%, bee venom in the ratio of 0.1%, bentonite in the ratio of 8%, jojoba oil in the ratio of 4%, shea butter in the ratio of 12%, propolis extract in the ratio of 0.3% and jasmine oil in the ratio of 0.5%.

12. A cream composition according to claim 10, comprising, the following by weight: deionized water in the ratio of 69.6%, glycerine in the ratio of 3%, NaCI in the ratio of 0.5%, royal jelly in the ratio of 1%, honey in the ratio of 1%, bee venom in the ratio of 0.1%, bentonite in the ratio of 7%, jojoba oil in the ratio of 5%, shea butter in the ratio of 12%, propolis extract in the ratio of 0.3% and jasmine oil in the ratio of 0.5%.

13. A cream composition according to claim 1, comprising, the following by weight: beeswax in the range of 10-15%, shea butter in the range of 10-25%, royal jelly in the range of 0.8-1.5%, olive oil in the range of 50-70%, honey in the range of 3-10%, bee venom in the range of 0.05-0.15%, propolis extract in the range of 0.1-0.5% and jasmine oil in the range of 0.5-1.5%.

14. A cream composition according to claim 13, comprising, the following by weight: beeswax in the ratio of 14.1%, shea butter in the ratio of 20%, royal jelly in the ratio of 1%, olive oil in the ratio of 58.5%, honey in the ratio of 5%, bee venom in the ratio of 0.1%, propolis extract in the ratio of 0.3% and jasmine oil in the ratio of 1%.

15. Production method of a cream composition according to claim 1, comprising, the following process steps:
a. Heating of an oil phase,
b. Cooling of the oil phase,
c. Adding a bee venom phase,
d. Homogenising, and
e. Adding propolis.

16. A production method according to claim 15, comprises a last step of placing a final product into containers.

17. A production method according to claim 15, wherein, in the “d” step, a mixing speed is 1500-2000 rpm.

18. A production method according to claim 15, wherein, in the “d” step, a mixing speed is 2000 rpm.

19. A production method according to claim 15, comprising, the following process steps so as to prepare said bee venom phase:
a. Mixing carob bean gum and water,
b. Heating the mixture until the gum melts,
c. Cooling the mixture when melting is realized,
d. Adding glycerin, honey, royal jelly and bee venom to the cooled mixture and mixing the same.

20. A production method according to claim 19, wherein the temperature is in the range of 85-95° C. in step “b”.

21. A production method according to claim 19, wherein the temperature is 90° C. in step “b”.

22. A production method according to claim 19, wherein the temperature is in the range of 45-50° C. in step “c”.

23. A production method according to claim 15, comprising, the process step heating stearyl alcohol, alcoholine GC, lipex shealight and jojoba oil with continuous stirring in a controlled manner until it reaches 50° C. in the heater so as to prepare said oil phase.

24. A production method according to claim 15, comprising the addition of vitamin E in addition to the bee venom phase in step “c”.

25. A production method according to claim 15, comprising the step of mixing deionized water, glycerin, NaCl, royal jelly, honey and bee venom until it dissolves to prepare said bee venom phase.

26. A production method according to claim 15, comprising, the following process steps so as to prepare said oil phase:
a. Heating by mixing bentonite, jojoba oil and shea butter,
b. Cooling the mixture after melting.

27. A production method according to claim 26, wherein the temperature is in the range of 35-45° C. in step “a”.

28. A production method according to claim 26, wherein the temperature is 40° C. in step “a”.

29. A production method according to claim 15, comprising jasmine oil in addition to propolis in step “e”.

30. A production method according to claim 15, comprising, the following process steps so as to prepare said oil phase:
a. Heating beeswax, shea butter and olive oil,
b. Cooling the oil phase after melting.

31. A production method according to claim 30, wherein the temperature is in the range of 60-80° C. in step “a”.

32. A production method according to claim 30, wherein the temperature is 70° C. in step “a”.

33. A production method according to claim 15, wherein said bee venom phase comprises royal jelly, honey and bee venom.
